(19) United States
(12) Patent Application Publication
(10) Pub. No.: US 2023/0066435 A1
(43) Pub. Date: Mar. 2, 2023

(54) ORTHOPEDIC SURGERY SYSTEM FOR SOFT TISSUE BALANCING AND IMPLANT PLANNING

(71) Applicant: Corin Limited, Cirencester (GB)

(72) Inventor: Christopher Plaskos, Plymouth, MA (US)

(73) Assignee: Corin Limited, Cirencester (GB)

(21) Appl. No.: 17/900,582

(22) Filed: Aug. 31, 2022

Related U.S. Application Data
(60) Provisional application No. 63/239,375, filed on Aug. 31, 2021.

(51) Int. Cl. A61B 34/10; A61B 34/30

(52) U.S. Cl. CPC A61B 34/10; A61B 34/30; A61B 2034/107; A61B 34/25

(57) ABSTRACT

A computer assisted orthopedic surgery system for soft tissue balancing and implant planning is provided. The system includes a three dimensional position tracking system, a robot, a display, and a computer. The computer is operatively in communication with the three dimensional position tracking system, the robot and the display. The computer includes a processor configured to acquire native gap data between a first bone and a second bone of a joint, simulate implant gap data between a first implant model on a first bone model of the first bone and a second implant model on a second bone model of the second bone of the joint based on an implant planning criteria to calculate a plurality of implant gap profiles, determine a best match of the plurality of implant gap profiles to the native gap profile to determine an optimized implant plan, and output the optimized implant plan.

### CROSS REFERENCE TO RELATED APPLICATIONS

The present application claims the benefit of U.S. Provisional Patent Application No. 63/239,375 filed Aug. 31, 2021 entitled Patient Specific Soft Tissue Balance and Implant Planning Joint Replacement System, the entire disclosure of which is hereby incorporated herein by reference.

### BACKGROUND OF THE DISCLOSURE

The present invention relates generally to a computer assisted orthopedic surgery system for joint replacement surgery and improving soft tissue balancing and implant positioning associated with such procedures.

Joint replacement surgery is performed on patients with degenerative joint diseases, such osteoarthritis and arthrosis, with the goals of relieving pain and restoring function, thus improving the quality of life for the patient. Although joint replacement surgery is exceedingly common, with approximately 700,000 knee replacement procedures performed annually in the U.S., it has been reported that a significant portion of patients (approximately one in five) are not satisfied with the results of their surgery. While this may be due to a number of factors, such as patient expectations, it is suspected that surgical technique related factors may play an important role in the number of cases that have less than optimal outcomes. In fact, several clinical studies have indicated that soft tissue related factors, such as instability and stiffness, are the leading cause for failure of total knee arthroplasty (TKA).

The act of achieving the appropriate soft-tissue tension and balance in joint replacement surgery is still regarded as somewhat of an art form by surgeons. This is partly because the act of assessing the tension in the soft tissues that surround a joint is largely a subjective process where the surgeon manually applies forces and moments to one side of the joint and observes the opening or compliance of the joint under the applied force by feel and by eye. Thus the assessment of soft tissue tension may vary depending on the surgeon performing the assessment, how they were trained, hold the limb by hand, and this may also vary from day to day, or from their left to right hand.

The standard of care in joint replacement surgery today is to use manual instrumentation which includes alignment rods, cutting blocks, provisional trial implants, and tensioning tools such as laminar spreaders or specifically designed manual spreaders. Robot and computer assisted surgery systems have been introduced in the late 90's and have been increasing in development and use. However, most systems currently on the market only partially address the soft tissue tensioning and balancing problem. Moreover, these systems still require a large number of instruments and provisional trial components to be available in the operating room.

Thus, there is still a need for a system and method for improving soft tissue balancing and implant planning for arthroplasty procedures. Such a need is satisfied by the present invention.

### BRIEF SUMMARY OF THE INVENTION

In accordance with an exemplary embodiment, the subject disclosure provides for a computer assisted orthopedic surgery system for soft tissue balancing and implant planning comprising: a three dimensional position tracking system; a robot; a display; and a computer operatively in communication with the three-dimensional position tracking system, the robot and the display, the computer including a processor configured to: acquire native gap data between a first bone and a second bone of a joint throughout a range of motion from the robot to calculate a native gap profile, simulate implant gap data between a first implant model on a first bone model of the first bone and a second implant model on a second bone model of the second bone of the joint throughout a range of motion based on an implant planning criteria to calculate a plurality of implant gap profiles each based on a unique implant planning criteria, determine a best match of the plurality of implant gap profiles to the native gap profile to determine an optimized implant plan, and output the optimized implant plan on the display.

In accordance with an aspect, the processor is further configured to acquire the native gap data from the robot as the robot applies a distraction force between the first and second bones of the joint. In accordance with an aspect, the distraction force is about 70 to 90 Newtons. In accordance with an aspect, the gap data includes gap spacing, anterior-posterior translation, anterior-posterior rollback, and/or internal-external rotation. In accordance with an aspect, the native gap profile is a laxity profile. In accordance with an aspect, the implant planning criteria is an implant type, implant size, and/or implant position. In accordance with an aspect, the implant position is an anterior-posterior position, a medial-lateral position, a varus-valgus position, an internal-external rotation, and/or a flexion position. In accordance with an aspect, the best match of the plurality of implant gap profiles to the native gap profile is determined based on a predefined flexion angle, a predefined range of motion, and/or a minimization function of an area between the implant gap profile and the native gap profile. In accordance with an aspect, the processor is further configured to adjust the native gap data based on bone quality. In accordance with an aspect, the bone quality includes cartilage data and/or bone erosion data. In accordance with an aspect, the optimized implant plan includes an implant size and implant position for an implant on at least one of the first and second bones of the joint.

In accordance with another exemplary embodiment, the subject disclosure provides for a method for patient-specific soft tissue balancing and implant planning comprising the steps of: acquiring native gap data between a first bone and a second bone of a joint throughout a range of motion and calculating a native gap profile; simulating implant gap data between a first implant model on a first bone model of the first bone and a second implant model on a second bone model of the second bone of the joint throughout a range of motion based on an implant planning criteria and calculating a plurality of implant gap profiles each based on a unique implant planning criteria; determining a best match of the plurality of implant gap profiles to the native gap profile to determine an optimized implant plan; and outputting the determined optimized implant plan to a display.

In accordance with an aspect, the step of acquiring native gap data includes applying a distraction force between the first and second bones of the joint as the native gap data is acquired. In accordance with an aspect, the method further comprising adjusting the native gap profile based on bone quality. In accordance with an aspect, the bone quality includes cartilage data and/or bone erosion data. In accordance with an aspect, the implant planning criteria is an implant type, implant size, and/or implant position. In accordance with an aspect, the implant position is an anterior-posterior position, a medial-lateral position, varus-valgus position, internal-external rotation, and/or flexion position. In accordance with an aspect, the best match of the plurality of implant gap profiles to the native gap profile is determined based on a predefined flexion angle, a predefined range of motion, and/or a minimization function of an area between implant gap profile and the native gap profile.

In accordance with an exemplary embodiment, the subject disclosure provides for a computer assisted orthopedic surgery system for soft tissue balancing and implant planning utilizing patient specific data e.g., patient specific gap data between bones of a joint.

### BRIEF DESCRIPTION OF THE SEVERAL VIEWS OF THE DRAWINGS

The foregoing summary, as well as the following detailed description of the exemplary embodiments of the invention, will be better understood when read in conjunction with the appended drawings. For the purpose of illustrating the invention, there are shown in the drawings exemplary embodiments. It should be understood, however, that the exemplary embodiments are not limited to the precise arrangements and instrumentalities shown.

In the drawings:

FIG. 1 is a schematic diagram of a computer assisted orthopedic surgery system in accordance with an exemplary embodiment of the subject disclosure;

FIG. 2 is a schematic drawing of an exemplary robot in accordance with an exemplary embodiment of the subject disclosure;

FIG. 3 is a perspective view of an exemplary orthopedic distraction device in accordance with an exemplary embodiment of the subject disclosure;

FIG. 4 is perspective view of an exemplary orthopedic distraction device used on a knee joint in accordance with an exemplary embodiment of the subject disclosure;

FIG. 4A is a schematic side view of a knee joint showing a native gap;

FIG. 5 is a front view of a display illustrating native gap profiles in accordance with an exemplary embodiment of the subject disclosure;

FIG. 5A is a front view of a display illustrating a native gap profile and an implant gap profile;

FIG. 6 is a front view of a display illustrating implant gap profiles in accordance with an exemplary embodiment of the subject disclosure;

FIG. 7 is a schematic side view of a knee joint showing an implant gap; and

FIG. 8 is a front view of a display illustrating an overlay of native and implant gap profiles in accordance with an exemplary embodiment of the subject disclosure.

### DETAILED DESCRIPTION OF THE DISCLOSURE

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the center of a body. The term “proximal” shall mean closer towards the center of a body and/or away from the “distal” end. The words “inwardly” and “outwardly” refer to directions toward and away from, respectively, the geometric center of the identified element and designated parts thereof. Such directional terms used in conjunction with the following description of the drawings should not be construed to limit the scope of the subject disclosure in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, or ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout this disclosure, various aspects of the subject disclosure can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the exemplary embodiments of the subject disclosure may be combined in any suitable manner in one or more embodiments. One skilled in the relevant art will recognize, in light of the description herein, that the present disclosure can be practiced without one or more of the specific features or advantages of a particular exemplary embodiment. In other instances, additional features and advantages may be recognized in certain embodiments that may not be present in all exemplary embodiments of the subject disclosure.

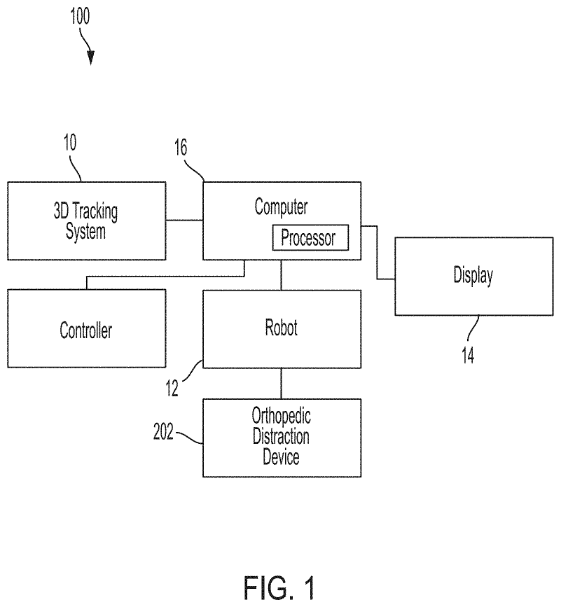

In accordance with an exemplary embodiment, the subject disclosure provides for a computer assisted orthopedic surgery system 100 (FIG. 1) that includes a three dimensional position tracking system 10, a robot 12, a display 14, and a computer 16. The computer is operatively in communication with the three dimensional position tracking system, the robot, and the display. Exemplary three dimensional position tracking systems, robots, and displays applicable to the exemplary embodiments of the subject disclosure are disclosed e.g., in U.S. Pat. No. 10,321,904, the entire disclosure of which is hereby incorporated by reference in its entirety for all purposes.

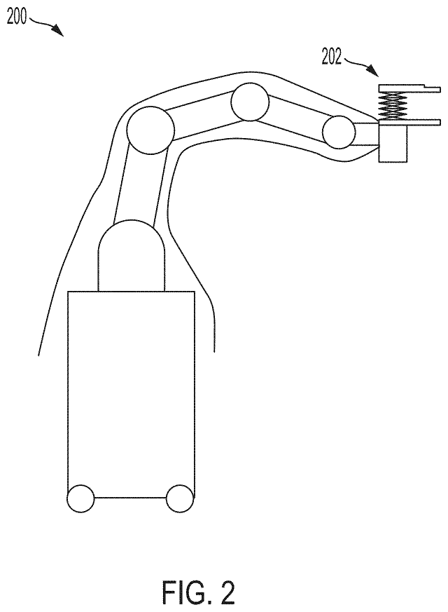

The robot 12 can include a robotic arm 200 (FIG. 2) controllable by the computer for performing various operations. Exemplary robotic arms and/or robotic systems applicable to the present exemplary embodiments are further disclosed in U.S. Pat. Nos. 10,321,904, 9,002,426 and 7,747,311, the entire disclosures of which are hereby incorporated by reference herein in their entirety for all purposes.

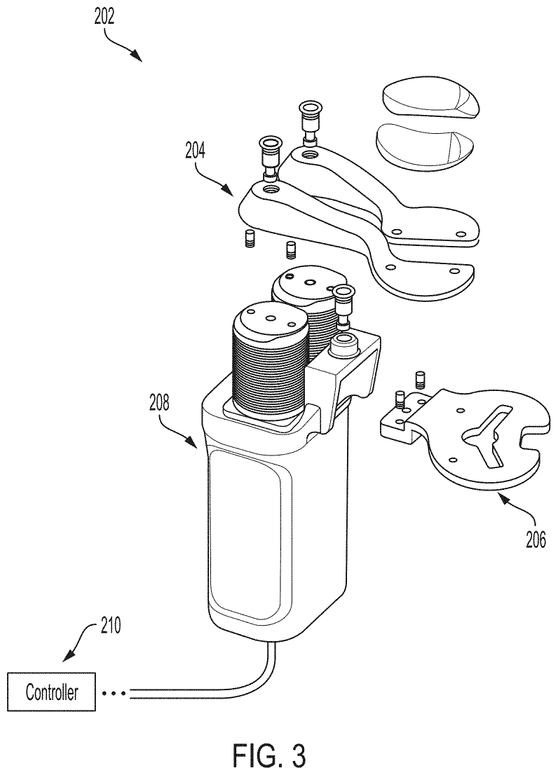

Referring to FIGS. 2 and 3, the robotic arm 200 can also include an orthopedic distraction device 202. The orthopedic distraction device 202 includes an upper paddle 204 for engaging a first bone of a joint, a lower paddle 206 for engaging a second bone of the joint, a displacement mechanism 208 operable to displace the upper paddle relative to the lower paddle, and a controller 210 operatively in communication with the displacement mechanism. The controller 210 is configured to apply displacement forces including varying displacement forces, to displace the upper paddle from the lower paddle based on a relative position between the first and second bones of the joint. Exemplary orthopedic distraction devices applicable to exemplary embodiments of the subject disclosure are disclosed in U.S. Pat. No. 10,321,904.

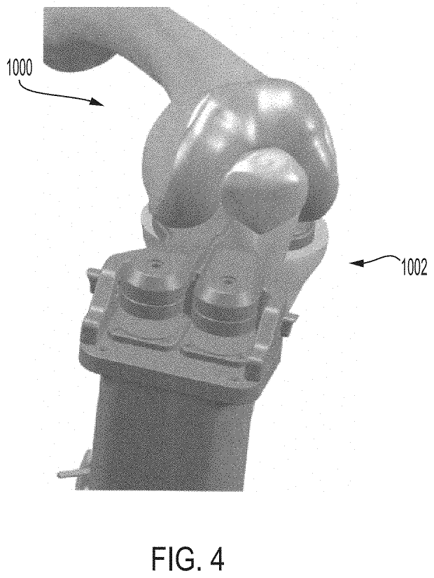

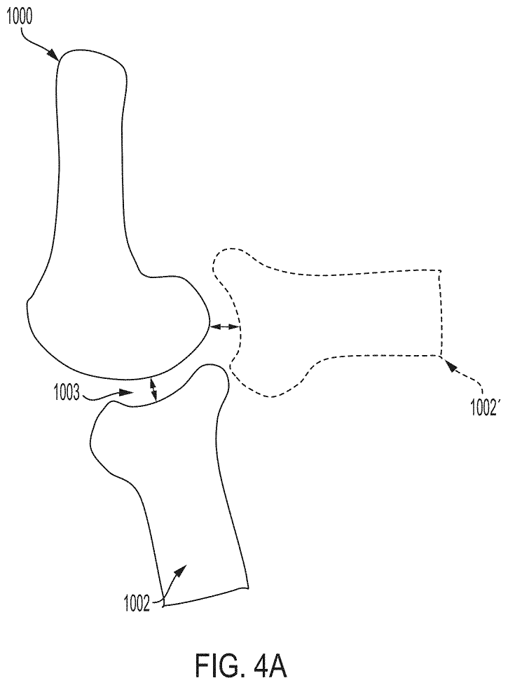

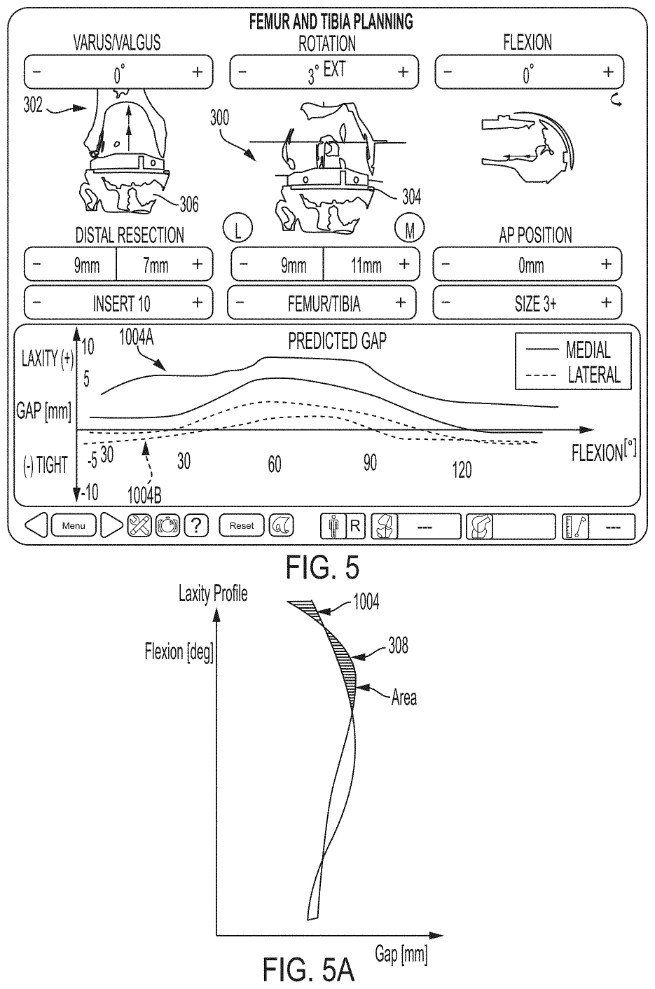

Referring to FIGS. 4 and 4A, the orthopedic distraction device 202 is configured to measure displacements or gaps between a first bone 1000 and a second bone 1002 of a joint, such as a knee joint. The gap between the bones of a joint of a patient is a native gap 1003. The native gaps can also be measured as the joint is taken throughout a range of motion, e.g., from −15 degrees to 140 degrees of flexion (see also FIG. 4A as second bone 1002 moves between a first position and a second position, referenced as 1002′. The gap data between the first and second bones of the joint of a patient is native gap data that is acquired by the computer. This native gap data is used to calculate a native gap profile 1004 (FIG. 5A) of the subject patient. The native gap profile can be a laxity profile, such as a gap v. flexion curve.

The computer processor is also configured to adjust the native gap data based on bone quality. For example, the native gap data can be adjusted and/or an adjustment factor applied based on bone quality that includes cartilage data and/or bone erosion data.

In other words, an adjustment factor can be incorporated into the native gap data to account for bone and/or cartilage wear. This adjustment factor can be based on pre-operative bone deformity (e.g., varus deformity, which in a knee joint will have wear on a medial condyle of the femur and/or tibia, and valgus deformity, which in a knee joint will have wear on a lateral condyle of the femur and/or tibia). That is, the adjustment factor can be based on patient specific pre-operative images of a patient's joint.

Cartilage and/or bone wear can change the native gap measurements between first and second bones of a joint throughout its range of motion because material has eroded from the cartilage and/or bone surface and the distance between the two bones has changed as a result. Thus, the computer processor is configured to compensate for such wear of the natural or native bone/joint and native gaps by e.g., 1) having a surgeon visually estimating and entering a compensation factor or adjustment to the native gap data directly into computer, 2) using statistical shape models (SSM) developed with healthy joints, 3) using shape fitting (spheres or circles, profile/tangent matching, etc.) to compensate for such wear, and/or 4) using estimates from patient specific pre-operative imaging to compensate for cartilage and/or bone wear in the native gap measurement. Alternatively, the system can be configured to have a user identify cartilage and/or worn areas on a bone intra-operatively, and then have the computer reconstruct bone and/or cartilage wear by any of the above discussed methods.

The system 100 can use pre-operative imaging (e.g., CT or MRI, or 2D to 3D SSM Xray techniques) of the subject joint to identify and segment out bone surfaces that require a compensation. The patient bone models can then be registered to the patient bones intra-operatively and the surfaces of the patient bones can be used to calculate gap profiles throughout a range of motion.

Additionally, the system can be configured to compensate for bone/cartilage surface wear based on known relationships, such as those disclosed in Nam, D., Lin, K. M., Howell, S. M. et al. Femoral bone and cartilage wear is predictable at 0° and 90° in the osteoarthritic knee treated with total knee arthroplasty. Knee Surgery Sports Traumatology Arthroscopy 22, 2975-2981 (2014). https://doi.org/10.1007/s00167-014-3080-8, the entire disclosure of which is hereby incorporated by reference herein in its entirety for all purposes.

The gap data of the native gap data and that of the implant gap data, as further described below, can include gap spacing, anterior-posterior translation, anterior-posterior rollback, and/or internal-external rotation.

The orthopedic distraction device 202 can be configured to measure gap data between the first and second bones of the joint of the patient before any bone resections are made to either of the first or second bones of the joint, or after a bone resection has been made to one of the first and second bones of the joint.

To address soft tissue balancing of the joint, the orthopedic distraction device 202 is configured to measure gaps between the first and second bones of the joint as the orthopedic distraction device applies a distraction force between the first and second bones of the joint. The applied distraction force can be from about 50 to 150 Newtons, preferably about 70 to 90 Newtons, including 55, 60, 65, 75, 80, 85, 95, 100, 105, 110, 120, 130, and 140 Newtons. The applied distraction force can be a constant force applied as the gap data is measured throughout a range of motion of the joint. Additionally, as applied to a knee joint, the applied distraction force can be independently applied to each of the medial and lateral condyles of the femur.

While the foregoing orthopedic distraction device has been described herein with respect to a knee joint, the orthopedic distraction device can also be configured to measure displacement or gaps between other joints of the body, such as the hip joint, shoulder joint, and elbow joint.

The computer 16 includes the display, a processor 18, and a non-transitory computer-readable medium or memory having stored thereon at least one of a first bone model and a second bone model of a joint, such as a femoral bone model and a tibial bone model of a knee joint, a first implant model associated with the first bone model and a second implant model associated with the second bone model, such as a femoral knee implant model and a tibial knee implant model.

The computer may also be configured to generate the first and/or second bone models of the joint. When generating the first and/or second bone models of the joint, intra-operatively generated data for the patient's joint bones can be used and incorporated into the bone models. Intraoperative data of the patient's joint bones can be obtained e.g., by passing a probe through the cartilage of the joint and taking surface measurements of the subject bone surface (e.g., both the first and second bones of a joint) below or behind the cartilage surrounding the joint bone surface. This bone surface data is then calculated/incorporated into the first and second bone models of the joint to calculate native gap data.

The computer also includes software or computer instructions for planning joint replacement procedures, including algorithms for planning the position of implants on the patients bones based off of bone morphology data, as further disclosed in U.S. Pat. No. 10,321,904.

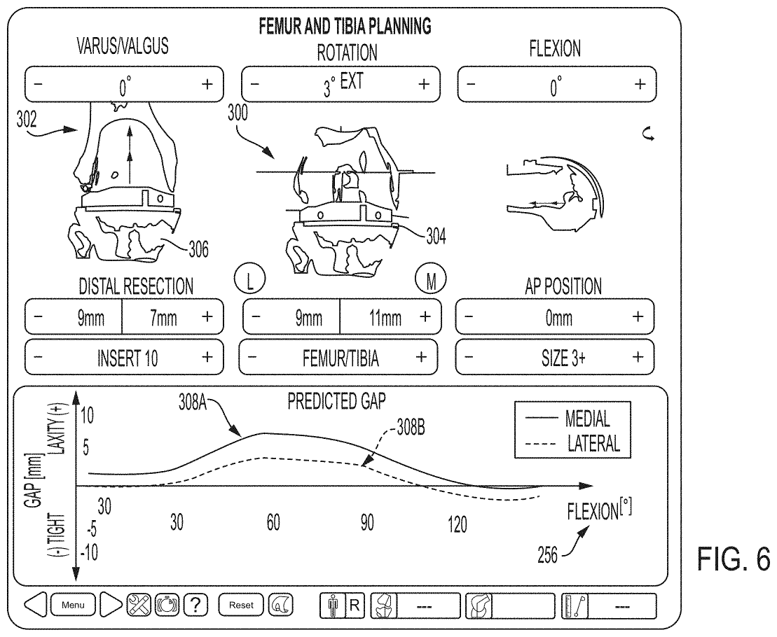

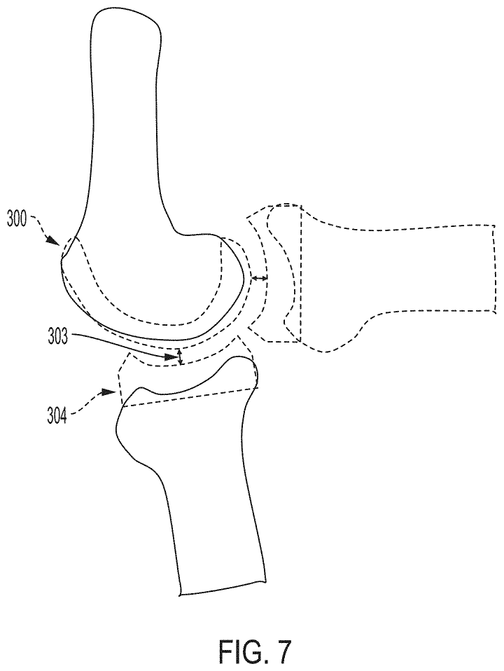

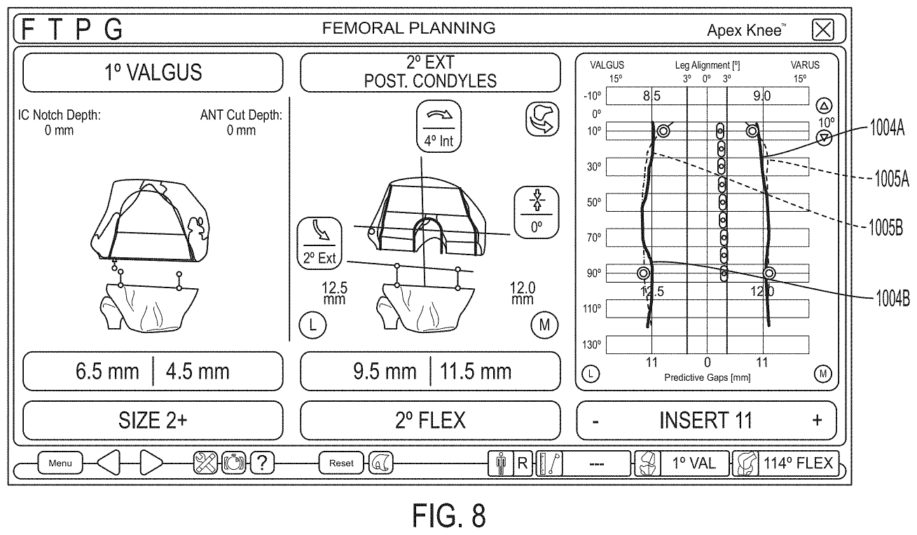

Referring to FIG. 6, the memory also includes computer program instructions executable by the processor to cause the computer to simulate implant gap data between a first implant model 300 on a first bone model 302 of the first bone and a second implant model 304 on a second bone model 306 of the second bone of the joint throughout a range of motion based on an implant planning criteria to calculate a plurality of implant gap profiles 308 each based on a unique implant planning criteria. The simulated implant gap data is also configured to be simulated with the same distraction force as that applied when acquiring native gap data of the joint of the patient in which the simulated gap data is to be evaluated against. The implant gap data is based on an implant gap 303 (FIG. 7) between first and second implant models positioned on first and second bone models respectively, and/or first and second implants positioned on first and second bones of a joint, respectively. FIG. 8 illustrates implant gap profiles overlayed with native gap profiles, e.g., a medial implant gap profile 1005A for a medial condyle of a knee implant, and a lateral implant gap profile 1005B for a lateral condyle of a knee implant.

In connection with simulated implant gap data of a knee joint, the implant gap profile is calculated for each of the medial and lateral condyles. That is, the processor calculates an implant gap profile of a medial condyle 308A and an implant gap profile of a lateral condyle 308B.

In simulating the implant gap data, the first and second implant models are evaluated or positioned on the respective first and second bone models based on implant planning criteria. The implant planning criteria can be an implant type for the implant models, an implant size for the implant models, and/or an implant position of the implant models on the bone models. The implant type can be that of e.g., a femur, a tibia, a tibial construct that includes a tibial baseplate and bearing insert, a unicompartmental femur, unicompartmental tibia, a hip stem, or an acetabular cup, or other implant types for a joint.

The implant position applicable to the implant planning criteria can be e.g., an anterior-posterior position, a medial-lateral position, a varus-valgus position, an internal-external rotation, and/or a flexion position. Additionally, the implant position can be an initial position for an implant model on a bone model that matches or is an equivalent to a position of the native bone of the patient. That is, various anatomical features of the implant model and bone model are initially positioned to match the anatomical features of the native bone of the patient which is the subject of an assessment by the computer assisted orthopedic surgery system. Referring to FIG. 8, the various implant planning criteria can be adjusted via inputs by a user on a display for the implant model such as implant size, flexion degree, varus-valgus position, internal-external rotation and then outputted as the implant gap profile in real time. Each implant gap profile is calculated based on a unique implant planning criteria. That is, each of the plurality of implant gap profiles generated by the computer is based on a unique set of implant planning criteria.

Referring to FIG. 5, after an implant gap profile 308 is calculated, it is displayed on the display overlapping the native gap profile 1004 (FIG. 5A). FIG. 5 illustrates a native gap profile 1004A for a medial side of a knee joint and a native gap profile 1004B for a lateral side of a knee joint. Thereafter, the computer processor determines a best match of the plurality of implant gap profiles to the native gap profile of the patient to determine an optimized implant plan. The processor can determine the best match based on a predefined flexion angle (such as 0, 45, or 90 degrees flexion), a predefined range of motion (such as 0 to 90 degrees flection), and/or a minimization function of an area between the implant gap profile and the native gap profile.

The optimized implant plan can include an implant size and/or an implant position for an implant on at least one of the first and second bones of the joint of the patient. The optimized implant plan is displayed on the display.

In other words, the system includes software executable to automatically determine a best match of the implant gap profile to the native gap profile. The software can include algorithms configured to minimize the area between the native gap profiles and planned or predicted gap profiles of the implants throughout a range of motion, assign weighting to different ranges of flexion (e.g., mid-flexion weighted greater than near full extension or deep flexion, or vice versa). The system is also configured to respect overall and/or component alignment limits (e.g., varus/valgus limits of a joint), and display and match various joint angles, such as LDFA (lateral distal femoral angle), and MPTA (medial proximal tibial angle).

In accordance with another exemplary embodiment of the subject disclosure, the present disclosure provides for a method for patient-specific soft tissue balancing and implant planning. The method includes the steps of: acquiring native gap data between a first bone and a second bone of a joint throughout a range of motion and calculating a native gap profile; simulating implant gap data between a first implant model on a first bone model of the first bone and a second implant model on a second bone model of the second bone of the joint throughout a range of motion based on an implant planning criteria and calculating a plurality of implant gap profiles each based on a unique implant planning criteria; determining a best match of the plurality of implant gap profiles to the native gap profile to determine an optimized implant plan; and outputting the determined optimized implant plan to a display.

In accordance with an aspect, the step of acquiring native gap data includes applying a distraction force between the first and second bones of the joint as the native gap data is acquired. In accordance with another aspect, the method further comprises adjusting the native gap profile based on bone quality. In accordance with an aspect, the bone quality includes cartilage data and/or bone erosion data. In accordance with an aspect, the implant planning criteria is an implant type, implant size, and/or implant position. In accordance with an aspect, the implant position is an anterior-posterior position, a medial-lateral position, varus-valgus position, internal-external rotation, and/or flexion position. In accordance with an aspect, the best match of the plurality of implant gap profiles to the native gap profile is determined based on a predefined flexion angle, a predefined range of motion, and/or a minimization function of an area between implant gap profile and the native gap profile.

It will be appreciated by those skilled in the art that changes could be made to the various exemplary embodiments and aspects described above without departing from the broad inventive concept thereof. It is to be understood, therefore, that the subject application is not limited to the particular exemplary embodiments and aspects disclosed, but it is intended to cover modifications within the spirit and scope of the subject application as defined by the appended claims.

I/we claim:

1. A computer assisted orthopedic surgery system for soft tissue balancing and implant planning comprising:
a three dimensional position tracking system;
a robot;
a display; and
a computer operatively in communication with the three dimensional position tracking system, the robot and the display, the computer including a processor configured to:
acquire native gap data between a first bone and a second bone of a joint throughout a range of motion from the robot to calculate a native gap profile,
simulate implant gap data between a first implant model on a first bone model of the first bone and a second implant model on a second bone model of the second bone of the joint throughout a range of motion based on an implant planning criteria to calculate a plurality of implant gap profiles each based on a unique implant planning criteria,
determine a best match of the plurality of implant gap profiles to the native gap profile to determine an optimized implant plan, and
output the optimized implant plan on the display.

2. The computer assisted orthopedic surgery system of claim 1, wherein the processor is further configured to acquire the native gap data from the robot as the robot applies a distraction force between the first and second bones of the joint.

3. The computer assisted orthopedic surgery system of claim 2, wherein the distraction force is about 70 to 90 Newtons.

4. The computer assisted orthopedic surgery system of claim 1, wherein the gap data includes gap spacing, anterior-posterior translation, anterior-posterior rollback, and/or internal-external rotation.

5. The computer assisted orthopedic surgery system of claim 1, wherein the native gap profile is a laxity profile.

6. The computer assisted orthopedic surgery system of claim 1, wherein the implant planning criteria is an implant type, implant size, and/or implant position.

7. The computer assisted orthopedic surgery system of claim 6, wherein the implant position is an anterior-posterior position, a medial-lateral position, a varus-valgus position, an internal-external rotation, and/or a flexion position.

8. The computer assisted orthopedic surgery system of claim 1, wherein the best match of the plurality of implant gap profiles to the native gap profile is determined based on a predefined flexion angle, a predefined range of motion, and/or a minimization function of an area between the implant gap profile and the native gap profile.

9. The computer assisted orthopedic surgery system of claim 1, wherein the processor is further configured to adjust the native gap data based on bone quality.

10. The computer assisted orthopedic surgery system of claim 9, wherein the bone quality includes cartilage data and/or bone erosion data.

11. The computer assisted orthopedic surgery system of claim 1, wherein the optimized implant plan includes an implant size and implant position for an implant on at least one of the first and second bones of the joint.

12. A method for patient-specific soft tissue balancing and implant planning comprising the steps of:
acquiring native gap data between a first bone and a second bone of a joint throughout a range of motion and calculating a native gap profile;
simulating implant gap data between a first implant model on a first bone model of the first bone and a second implant model on a second bone model of the second bone of the joint throughout a range of motion based on an implant planning criteria and calculating a plurality of implant gap profiles each based on a unique implant planning criteria;
determining a best match of the plurality of implant gap profiles to the native gap profile to determine an optimized implant plan; and
outputting the determined optimized implant plan to a display.

13. The method of claim 12, wherein the step of acquiring native gap data includes applying a distraction force between the first and second bones of the joint as the native gap data is acquired.

14. The method of claim 12, further comprising adjusting the native gap profile based on bone quality.

15. The method of claim 14, wherein the bone quality includes cartilage data and/or bone erosion data.

16. The method of claim 12, wherein the implant planning criteria is an implant type, implant size, and/or implant position.

17. The method of claim 16, wherein the implant position is an anterior-posterior position, a medial-lateral position, varus-valgus position, internal-external rotation, and/or flexion position.

18. The method of claim 12, wherein the best match of the plurality of implant gap profiles to the native gap profile is determined based on a predefined flexion angle, a predefined range of motion, and/or a minimization function of an area between implant gap profile and the native gap profile.
